(19) United States
(12) Patent Application Publication
(10) Pub. No.: US 2023/0200700 A1
(43) Pub. Date: Jun. 29, 2023

(54) SYSTEMS AND METHODS FOR USING BIOIMPEDANCE TO DETERMINE MUSCLE ACTIVITY AND RELATED PROCESSES

(71) Applicant: Charles Zaloom iV, Encinitas, CA (US)

(72) Inventor: Charles Zaloom IV, Encinitas, CA (US)

(21) Appl. No.: 18/087,134

(22) Filed: Dec. 22, 2022

Related U.S. Application Data
(60) Provisional application No. 63/407,026, filed on Sep. 15, 2022.
(60) Provisional application No. 63/293,661, filed on Dec. 24, 2021.

(51) Int. Cl. A61B 5/25; A61B 5/00

(52) U.S. Cl. CPC A61B 5/25; A61B 5/6801

(57) ABSTRACT

Systems and methods for using bioimpedance analysis to determine the condition of muscles are generally described.

### RELATED APPLICATIONS

This application claims priority under 35 U.S.C. § 119(e) to U.S. Provisional Application No. 63/293,661, filed Dec. 24, 2021, and entitled “Wearable Health Sensing Devices,” and to U.S. Provisional 63/407,026, filed Sep. 15, 2022, and entitled “Systems and Methods for Using Bioimpedance to Determine Muscle Activity and Related Processes,” each of which are incorporated herein by reference in its entirety for all purposes.

### TECHNICAL FIELD

Systems and methods for using bioimpedance analysis to determine the condition of muscles are generally described.

### BACKGROUND

Throughout the last decades, health monitoring technology has continuously evolved to become more compact, lighter, and less invasive, with the objective of becoming easier to use by users while maintaining their function. This has particularly attracted other uses beyond those found in conventional medical settings. Tracking muscular fatigue is one of these applications, yet the technology has failed to attract a significant number of consumers in this area. One of the reasons for this failure may be the inability to be integrate this complex technology into everyday textiles. Everyday textiles may be worn by users exhibiting motion in a variety of directions and orientations, which have conventionally be unsuited for the complex technologies on the market (which may “fall off” often due to the variety of motions/directions/orientations of the user).

One particular tracking metric would be to measure muscular fatigue. However, accurately measuring muscular fatigue also presents multiple challenges due to the intricate nature of the activity, and, again, the inability to provide muscle activity monitoring without the technology removing from the user during the muscle activity.

### SUMMARY

Systems and methods for using bioimpedance analysis to determine the condition of muscles. The subject matter of the present disclosure involves, in some cases, interrelated products, alternative solutions to a particular problem, and/or a plurality of different uses of one or more systems and/or articles.

In one aspect, a wearable system configured to be worn by a user is described, the system comprising a plurality of reversibly detachable textile patches; a plurality of bioelectrical sensors integrated into each reversible detachable textile patch; a reversibly detachable computing unit; a power source in electrical communication with the plurality of bioelectrical sensors; and a plurality of conductive threads in electrical communication with the reversibly detachable computing unit and the plurality of bioelectrical sensors, wherein a first bioelectrical sensor of the plurality of bioelectrical sensors is a bioelectrical impedance analysis sensor, and wherein the wearable system is configured to collect a plurality of determinable signals from the plurality of bioelectrical sensors, wherein the plurality of determinable signals are correlated with a physiological health metric of the user.

In another aspect, a wearable system for sensing muscle fatigue is described, the system comprising a plurality of textile patches; a plurality of bioelectrical sensors integrated into the plurality of textile patches, wherein the textile patches are configured to be integrated into a garment; a computing unit; and a plurality of conductive threads electrically connecting the computing unit and the plurality of textile patches.

In another aspect, a wearable system for sensing muscle fatigue is described, the system comprising a plurality of textile patches; a plurality of conductive textile threads, wherein the conductive textile threads are incorporated into the textile patches, and wherein the conductive threads are capable of applying an oscillatory electrical signal to muscles through at least a portion of a skin surface.

In yet another aspect, a method for determining muscle fatigue is described, the method comprising providing a plurality of bioelectrical sensors integrated into the plurality of textile patches to a surface of skin; applying an oscillatory electric signal through the skin and across one or more muscles; determining a bioimpedance of the oscillatory electric signal; determining a state of the one or more muscles based, at least in part, on the determining of the bioimpedance.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic diagram of a wearable system comprising textile patches and a bioelectrical sensor, according to some embodiments;

FIG. 1B is a schematic diagram of a wearable system comprising a bioelectrical sensor and a computing unit, according to some embodiments;

FIG. 2 schematically illustrate a wearable garment integrated into a textile garment depicting textile patches, conductive threads, and a computing unit, according to some embodiments,

FIG. 3 schematically illustrates computing unit depicting a printed circuit board, an inductive wireless charging coil, a power source, a wireless interface for communication, a textile interfacing connector, and clip for attachment/detachment of the computing unit, according to some embodiments,

FIG. 4 schematically illustrates a textile patch depicting a conductive thread, a dry electrode, a motion sensor, and a temperature sensor, according to some embodiments,

FIG. 5 schematically illustrates a wearable garment in the form of an undergarment depicting a textile patch and a computing unit, according to some embodiments,

FIG. 6 schematically illustrate a wearable garment in the form of a compressive sleeve depicting a textile patch and a computing unit, according to some embodiments.

### DETAILED DESCRIPTION

Systems and methods for wearable health monitoring and motion tracking systems are described herein. The wearable health monitoring and motion tracking systems may comprise a plurality of bioelectrical sensors integrated into a plurality of textile patches, conductive thread, a computing unit, and a power source. Methods for determining physiological metrics are also described herein. The wearable system described herein may be worn by a user and may provide physiological metrics and/or physical motion information, such as muscle activity (e.g., fatigue) of a user).

The present disclosure relates to wearable health monitoring systems and/or methods including a wearable bioelectric impedance analysis sensor. In some embodiments, the wearable health monitoring systems described herein may be useful for determining physiological health metrics, including but not limited to health warnings (e.g., muscle fatigue), performance advise, hydration status, sleep quality, and the like. The wearable health monitoring systems may be worn by a user (e.g., an athlete) and may be used to capture different metrics (e.g., electrical signals, temperature, motion, etc.) and/or provide a diagnosis (e.g., muscle fatigue). In some embodiments, the user may be provided with additional information in the form of a pre-diagnosis that may prevent certain health problems such as muscular injuries, excessive fatigue, etc. The wearable health monitoring system may be used, in some embodiments, to obtain continuous information of the user by being integrated into everyday garments (e.g., a t-shirt, pants, armband). Such embodiments may be useful for a variety of reasons, e.g., for tracking certain physiological metrics and/or conditions with simple, non-invasive components.

The present disclosure describes systems and methods to integrate health monitoring devices into wearable garments. Health monitoring devices integrated into garments may come in different shapes and sizes, may be used for a variety of applications, and/or may offer many advantages over similar devices. For example, in some cases, muscle activity may be tracked in different parts of a user advantageously without sacrificing comfortability and/or restricting movement. These advantages may be attributed, in some cases, to enhanced flexibility of components used in the wearable garment. In some embodiments, flexibility may be advantageous for other reasons. For example, in some cases, flexible sensors (e.g., textile-based sensors) used for measuring muscular activity may be useful for improving the acquisition of signals by maximizing skin contact as the skin flexes/relaxes with the skin, the flexible sensor may also flex (without being damaged).

It has been recognized and discovered, as described in more detail below, by the present disclosure that bioelectrical impedance measurements may be acquired using a wearable system to obtain physiological metrics and motion tracking data. In some embodiments, the wearable system described herein comprises a reversibly detachable computing unit and a reversibly detachable textile patches, which may, in some cases, advantageously reduce the monetary cost of compatible garments as the compatible garments are not required, in some embodiments, to be fabricated along with the textile patches and/or computing units. Hence, the compatible garment may comprise a run-of-the mill t-shirt, a sophisticated dress, compression shorts, biking shorts, basketball shorts, a back brace, and/or girdle (without limitation). In some embodiments, the reversibly attached components (e.g., the reversibly detachable textile patches) may be attached, de-attached, and/or reattached to any one of these compatible garments (e.g., detached from a t-shirt and placed on a sophisticated dress; detached from a sophisticated dress and reattached to the t-shirt). As a result, the garments may cost less to manufacture and/or may cost less for the user or an owner to purchase.

As another advantage, in some embodiments, the detachable textiles patches and/or computing units may be replaced individually without replacing the entirety of the garment once again reducing costs for the user or owner. For example, if the health monitoring system were within a t-shirt without detachable patches, the system would last a long as the t-shirt itself. However, with reversibly detachable textile patches, the health monitoring system (comprising detachable textile patches) may simply be detached from the worn t-shirt and reattached to a newer t-shirt. This may also, in some cases, improve the ability to launder more frequently the garments attached to the detachable components (e.g., the detachable textiles patches and/or computing units), as the components may simply be detached, the garment washed, and the components reattached.

In some embodiments, the wearable system comprises conductive thread(s) that transmits electrical signals from bioelectric sensor to a computing unit which may advantageously result in a wearable system that is conformable to the skin of the user and comfortable for the user to wear by providing more comfortable configurations for the wearable components separated from the computing portions of power storage portions (e.g., a battery pack).

The wearable system described herein may comprise, in some embodiments, a plurality of bioelectrical sensors that captures temperature information or motion information. In some such embodiments, the wearable system may advantageously produce motion tracking information in addition to physiological metrics. As another advantage, the wearable system may cross-correlate information from user inputs to measured data to produce several other metrics including, but not limited to, nutrition metrics, sleep quality metrics, injury prevention metrics, and/or muscular activity metrics. Other metrics may also be produced. In some embodiments, a computing unit can transmit the above-mentioned metrics to external devices (e.g. a smartphone, tablet, or computer) wirelessly. As another advantage, health warnings and guidelines can be delivered to the external device to notify the user of their physiological condition. As another advantage, the wearable system may detect musculoskeletal abnormalities, health abnormalities, etc. without the intervention of a physician or clinical staff. As it obtains a continuous stream of health-related data, the wearable system may detect sudden or concerning changes in physiological or dietary trends and notify the user.

Turning to the figures, specific non-limiting embodiments are described in further detail. It should be understood that the various systems, components, features, and methods described relative to these embodiments may be used either individually and/or in any desired combination as the disclosure is not limited to only the specific embodiments shown in the figures.

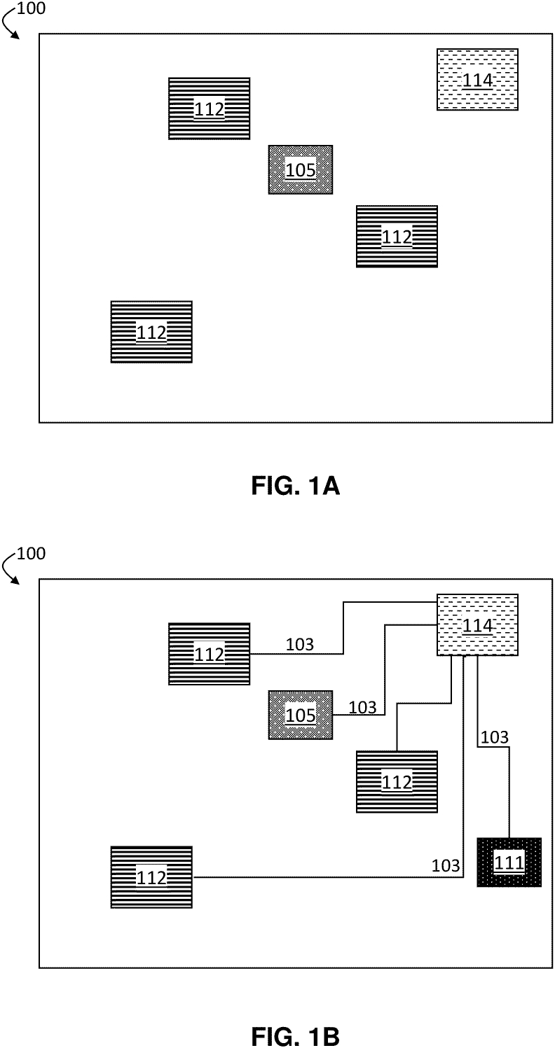

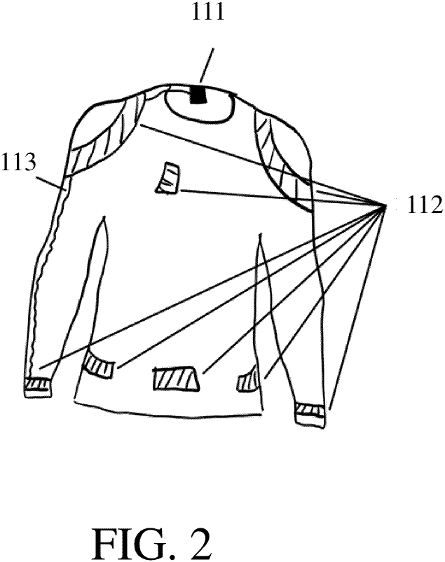

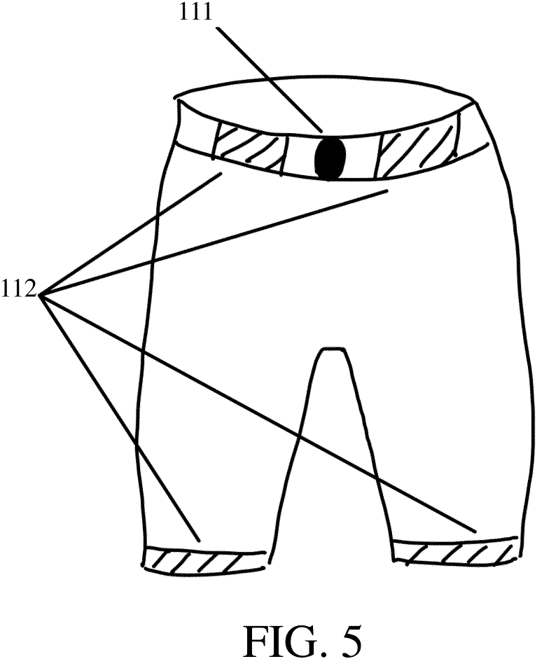

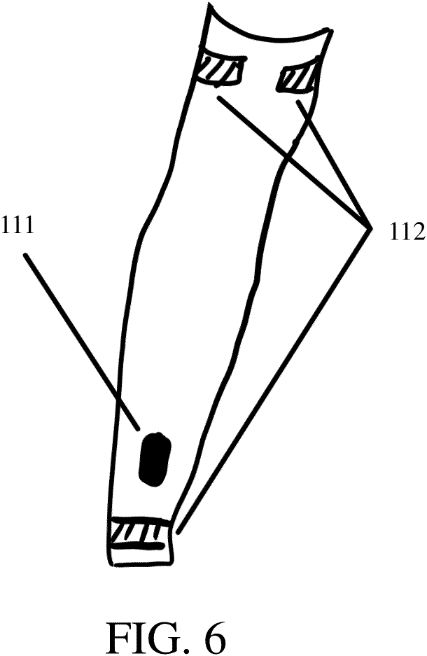

In some embodiments, a wearable system comprises a computing unit, textile patches, and conductive threads. For example, FIG. 1A shows a schematic diagram of an exemplary wearable system 100 that includes textile patches 112 connected to a power source 114. The wearable system may also include, in some cases, a bioelectrical sensor, which can be configured to send and/or receive a signal from the muscle(s) of a user. Other components may be included within the sample, in certain embodiments. For example, as shown in FIG. 1B, the wearable garment 100 may comprise conductive threads 103, which can connect to the power source or another component of the wearable system, along with a computing unit 111, which may process one or more algorithms for determining the condition of the muscle. FIG. 2 shows another exemplary wearable system in the form of a garment, wherein the garment is a shirt, comprising a plurality of textile patches 112 in various locations on the garment connected to a computing unit 111. As described above and herein, other garments are also possible. In some embodiments, conductive threads 103 provide an electrically conductive path to transmit electrical signals from each textile patch to the computing unit. In some embodiments, the garment can be a compressive shorts/pants or a compression sleeve as shown in FIG. 5 and FIG. 6.

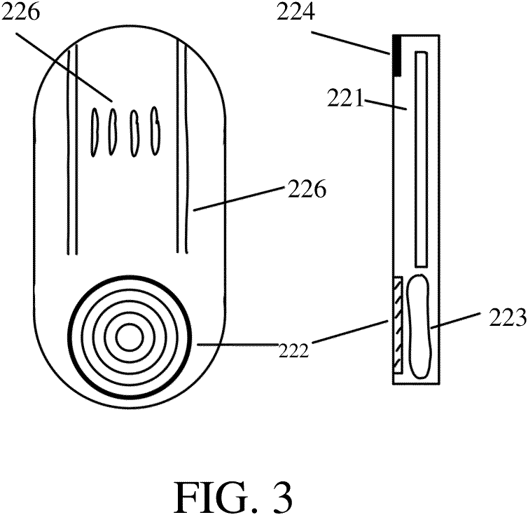

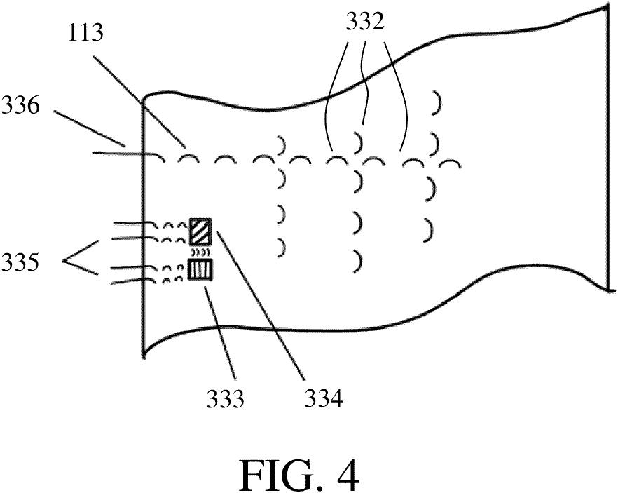

FIG. 3 depicts a computing unit in accordance with some embodiments which allows wireless recharge. The computing unit may contain a microcontroller and hardware for bioelectrical analysis. This unit can be integrated into the textile or made attachable through the use of the removable and/or reusable attachment mechanism. The computing unit contains the battery and wireless charging circuitry necessary to operate the device. Whether the computing unit is attachable or permanently integrated, it powers the entire completed textile when configured properly. The second unit exists in multiple forms but can be generalized as a textile patch that has either conductive thread or conductive fabric integrated into it. FIG. 3 also shows wireless inductive coils 222, chip rails 226, a battery 223 that powers the PCB board 221, one or more of which may be present, in some embodiments. Antenna 224 may be used, in some embodiments, to send and/or transmit a wireless signal (e.g., a Bluetooth) signal. Patches of such conductive thread or fabric may be woven, in some cases, to make dry electrodes (332) configured to performing bioelectrical measurements. In some embodiments, electrode pads are strategically placed to maximize skin contact based on a given article of clothing (e.g. shirt, pants, compression sleeve). The textile patches may be configured, in some embodiments, to have small and inexpensive electronic components such as accelerometers, gyros and thermometers integrated directly into them. An exemplary configuration shown in FIG. 4 includes such components. By delegating more expensive components involved in processing and circuitry inside the computing unit, the total cost per textile unit may be reduced, in some cases, thereby enabling clothing articles that can be manufactured cheaply with the computing device being a one-off per user.

In some embodiments, the computing unit contains the microcontroller and hardware for bioelectrical analysis. Such computing units may be integrated into the textile or made attachable through the use of a removable and reusable attachment mechanism. The computing unit contains the battery and wireless charging circuitry necessary to operate the device as a whole. Whether the computing unit is attachable or permanently integrated, it powers the entire completed textile when configured properly. The second unit exists in multiple forms but may be generalized as a textile patch that has either conductive thread or conductive fabric integrated into it. Patches of this conductive thread or fabric is woven, in some embodiments, to make dry electrodes 332 configured for performing bioelectrical measurements. These electrode pads may be, in some embodiments, strategically placed to maximize skin contact based on a given article of clothing (e.g. shirt, pants, compression sleeve). The textile patches may also be configured to have small and inexpensive electronic components such as accelerometers, gyros and thermometers integrated directly into them. This configuration is shown in FIG. 4. By delegating more expensive components involved in processing and circuitry inside the computing unit, the total cost per textile unit is reduced to enable clothing articles that may be manufactured cheaply with the computing device being a one-off per user.

In some embodiments, textile patches may be integrated into a garment and/or comprise a plurality of bioelectrical sensors and/or conductive thread. For example, FIG. 4 shows a textile patch comprising a plurality of bioelectrical sensors, wherein the bioelectric sensors may comprise one or more bioelectrical impedance analysis sensors, temperature sensors, and/or motion tracking sensors. In some embodiments, conductive threads 113 are used as electrodes 332 for the bioelectrical sensors to apply an electrical signal to the user’s skin. The conductive thread may allow for electrical communication with the computing unit, other bioelectrical sensors, and/or other textile patches. The conductive threads may be configured to apply oscillatory electrical signals to muscles through at least a portion of a skin surface.

In some embodiments, the computing unit may comprise a power source. For example, FIG. 3 shows a computing unit comprising a control module, an inductive wireless charging coil, a power source, a wireless interface, a textile interfacing connector, and/or a clip rail for reversible attachment/detachment. The power source may supply power to a plurality of bioelectrical sensors and/or the computing unit. The computing may be reversibly attached or detached from a garment with electrical communication engaged/disengaged via contact with the clip rail.

In some embodiments, the wearable system comprises a plurality of textile patches. Any textile patch in the plurality of textile patches may come in a different shape, placed in a specific or different orientation, and/or may be manufactured with at least one type of material. The textile patch may also be configured to be attachable to other textile-like components (e.g., a flexible electrode) as well as non-textile-like components (e.g., a rigid electrode). The textile patches may be also used as support (i.e., a substrate) to add any component that may be integrated into the wearable system. This may be useful for a variety of reasons, for example, to avoid certain components in the wearable system from moving around.

It should be understood that many materials may be used to fabricate a textile patch. Furthermore, combinations of different materials may be used as necessary to enhance certain properties of the textile patch, e.g., stretchability, comfortability, durability, etc. Some non-limiting examples of materials that could be used to fabricate a textile patch include silk, wool, linen, cotton, synthetic fibers (e.g., rayon, nylon, polyesters, etc.), and/or inorganic fibers (e.g., gold, glass fibers, etc.). Textile patches may also include one or more additional materials e.g., conductive materials, metals, polymers, ceramics, glass, etc.

In some embodiments, the textile patch may be configured to be integrated into a garment. The garment may be an item of clothing that may be worn by a user. Some examples of garment include tops (e.g., t-shirts), pants (e.g., jeans), armwear (e.g., gloves), jackets, robes, belts, skirts, sportswear, coats, jackets, masks, suits, footwear, undergarments, headgear (e.g., a hat), etc. In addition, the textile patch may be attached in a variety of ways into the garment, including mechanical and/or chemical adhesion, for example, by using stitches, glue, fasteners, hook and loop, or the like, without limitation.

In addition, in certain embodiments, a textile patch may not be strictly necessary for the wearable system. For instance, in some cases, certain components (e.g., bioelectrical sensors) may not need certain advantages offered by a textile patch (e.g., structural support) and may be attached directly to the garment.

In some embodiments, the textile patch may be permanently attachable. However, in other embodiments, the textile patches are detachable to and from (and, at least, to again) the garment. Permanently attachable textile patches may refer to a textile patch which is manufactured with the intention of remaining attached during the lifetime of the garment. On the other hand, detachable textile patches may be removed by the user. In some situations, having detachable textile patches may be useful for certain reasons, e.g., to replace or to modify the textile patch. In addition, detachable textile patches may be reversibly attachable and detachable, which may allow the textile patch to be repositioned, for example.

In some embodiments, a plurality of bioelectrical sensors may be used (e.g., to detect muscle activity, monitor muscle fatigue). In some embodiments, the bioelectrical sensors may be permanently attached (i.e., stitched) to the textile patch. However, in some cases, the bioelectrical sensors may be detachable from the textile patch. This may be advantageous for replacing the bioelectrical sensors when needed, for example. In some embodiments, the plurality of bioelectric sensors is configured to pass a current through tissue and measuring a response of the tissue, at least in part, based on the current (and/or change in the current). In some embodiments, the plurality of bioelectrical sensors is configured to measure a voltage related to muscular activation and measuring a response of the tissue, at least in part, on the measured voltage (and/or a change in the measured voltage).

In some cases, a first bioelectrical sensor and a second bioelectrical sensor of the plurality of bioelectrical sensors may be integrated into a textile patch. In some cases, the first bioelectrical sensor and the second bioelectrical sensor may have different functionalities (e.g., bioimpedance and temperature sensing). Nonetheless, the first bioelectrical sensor and the second bioelectrical sensor may not need to perform different functionalities.

In some embodiments, a first bioelectrical sensor of the plurality of bioelectrical sensors may comprise one or more bioelectrical impedance analysis sensors. The bioelectrical impedance analysis sensor may be configured to, in some embodiments, of applying and/or measuring a bioimpedance signal in bodily tissue to monitor different bodily functions and/or activities. Some of these bodily functions and/or activities may be electrochemical processes, electrical impedance, ionic movement, resistivity of individual components, or the like. The bioimpedance signal may comprise an alternative current (AC) signal, according to some embodiments. The AC signal is an electric current in the form of an oscillatory signal with tunable frequency and amplitude. Taking advantage of these parameters, the bioimpedance signal may be useful to measure different properties of bodily tissue. For example, at certain frequencies, solid components (e.g., cells and membranes) may have different electrical resistivity values, therefore, the bioimpedance signal may flow through the least resistive component (e.g., cells) which may provide information in relation to the ratio of cells to membranes. As another example, at certain frequencies, the AC signal may flow better through portions of a tissue with lower resistivity (e.g., a hydrated muscle) thus may be useful for determining certain properties (e.g., hydration) of the bodily tissue being analyzed. In addition, the frequency and amplitude may be tuned to distinguish between some of these bodily mechanisms and/or components described herein. Some examples of optimization routes may include tuning the frequency and/or amplitude of the AC signal. This may result in measurements with reduced noise, stronger signal, etc.

For embodiments in which an AC signal is generated and/or measured by the wearable system, each of these embodiments may have independently the same or different frequencies and amplitudes. In some embodiments, the AC signal may have a frequency of less than or equal to 10 kHz, less than or equal to 20 kHz, less than or equal to 30 kHz, less than or equal to 40 kHz, less than or equal to 50 kHz, less than or equal to 60 kHz, less than or equal to 70 kHz, less than or equal to 80 kHz, less than or equal to 90 kHz, less than or equal to 100 kHz, less than or equal to 110 kHz, less than or equal to 120 kHz, less than or equal to 130 kHz, less than or equal to 140 kHz, less than or equal to 150 kHz, less than or equal to 160 kHz, less than or equal to 170 kHz, less than or equal to 180 kHz, less than or equal to 190 kHz, or less than or equal to 200 kHz. In some embodiments, the AC signal may have a frequency of greater than or equal to 10 kHz, greater than or equal to 20 kHz, greater than or equal to 30 kHz, greater than or equal to 40 kHz, greater than or equal to 50 kHz, greater than or equal to 60 kHz, greater than or equal to 70 kHz, greater than or equal to 80 kHz, greater than or equal to 90 kHz, greater than or equal to 100 kHz, greater than or equal to 110 kHz, greater than or equal to 120 kHz, greater than or equal to 130 kHz, greater than or equal to 140 kHz, greater than or equal to 150 kHz, greater than or equal to 160 kHz, greater than or equal to 170 kHz, greater than or equal to 180 kHz, greater than or equal to 190 kHz, or greater than or equal to 200 kHz. Any combination of the above-referenced frequencies may also be possible (e.g. less than or equal to 100 kHz and greater than or equal to 10 kHz). Other ranges are possible as the disclosure is not so limited.

In some embodiments, the AC frequency may sweep from one frequency (e.g., a first frequency) to another frequency (e.g., a second frequency). In some embodiments, sweeping across several frequencies may be achieved, and may constitute a cycle. For example, in a first cycle, the AC frequency may move from a first frequency to a second frequency, and back to a first frequency. As another example, in a second cycle, the AC frequency may move from a third frequency to a fourth frequency; from a fourth frequency to fifth frequency; from a fifth frequency to a sixth frequency; and, optionally, from a sixth frequency back to the third frequency. Advantageously, using frequency sweeps may provide more bioimpedance data (e.g., each frequency may provide the same or different data, which may be statically helpful in generating high SNR data). Those skilled in the art will be capable of selecting an appropriate AC frequency and sweeping sequence in order to obtain AC signals for monitoring muscle activity based upon the teachings of this specification.

In some embodiments, the AC signal may have an amplitude of less than or equal less than or equal to 0.1 µA, less than or equal to 1 µA , less than or equal to 10 µA, less than or equal to 100 µA, less than or equal to 200 µA, less than or equal to 300 µA, less than or equal to 400 µA, , less than or equal to 500 µA, less than or equal to 600 µA, less than or equal to 700 µA, less than or equal to 800 µA, less than or equal to 900 µA, less than or equal to 1000 µA, less than or equal to 1100 µA, less than or equal to 1200 µA, less than or equal to 1300 µA, less than or equal to 1400 µA, or less than or equal to 1500 µA. In some embodiments, the AC signal may have an amplitude of greater than or equal 1500 µA , less than or equal to 1400 µA, greater than or equal to 1300 µA, greater than or equal to 1200 µA, greater than or equal to 1100 µA, greater than or equal to 1000 µA greater than or equal to 900 µA, greater than or equal to 800 µA, greater than or equal to 700 µA, greater than or equal to 600 µA, greater than or equal to 500 µA, greater than or equal to 400 µA, greater than or equal to 300 µA, greater than or equal to 200 µA, greater than or equal to 100 µA, greater than or equal to 10 µA, greater than or equal to 1 µA, or greater than or equal to 0.1 µA. Other ranges are possible. In some cases, the AC signal may have a constant amplitude. In other embodiments, the amplitude may change overtime.

In some embodiments, a second bioelectrical sensor of the plurality of bioelectrical sensors may be a sensor different that a bioimpedance sensor. For example, a second bioelectrical sensor of the plurality of bioelectrical sensors may be a temperature sensor 334 and/or a motion sensor. In some embodiments, the temperature sensor may be used to measure a temperature of the skin. Various contact temperature sensors 334, including but not limited to thermistors, thermocouples, semiconductor-based sensors, and resistance temperature detectors, are known in the art. In addition, beyond contact temperature sensors, non-contact temperature sensors may also be used. Non-contact temperature sensors may use radiation or convection to measure temperature. An example of a non-contact, radiation-based sensor is an infrared sensor.

Some examples of motion sensors 333 include gyroscopes, accelerometers, inclinometers, goniometers, etc. Some motion sensors may be configured to measure motion in one axis (e.g., a single-axis MEMS gyroscope), multiaxial motion, acceleration, etc., thus being capable of tracking body movements. Different motion sensors may be useful to distinguish between different type of movements (e.g., driving and walking). In addition, some motion sensors may be capable of recognizing other mechanical motions such as vibrations and mechanical shocks.

The data obtained from the first bioelectrical sensor and the second bioelectrical sensor may be cross-correlated. For example, the first bioelectrical sensor (e.g., a bioimpedance sensor) may generate a signal at a particular frequency (e.g., 5 Hz) that may be cross-correlated with another signal generated by the second bioelectrical sensor at the same frequency, thus the first bioelectrical sensor may work in parallel with the second bioelectrical sensor (e.g., a piezoelectric accelerometer) to produce cross-correlated data. As a result of this cross-correlation, certain metrics may be determined.

Two or more bioelectrical sensors of the plurality of bioelectrical sensors may be spaced at various distances. For instance, in some embodiments, two or more sensors may be spaced at a distance of greater than or equal to 1 nm, greater than or equal to 10 nm, greater than or equal to 100 nm, greater than or equal to 1 µm, greater than or equal to 10 µm, greater than or equal to 100 µm, greater than or equal to 1 mm, greater than or equal to 10 mm, greater than or equal to 100 mm, greater than or equal to 1 m, etc. In some embodiments, two or more bioelectrical sensors may be spaced at a distance of less than or equal to 1 m, less than or equal to 100 mm, less than or equal to 10 mm, less than or equal to 1 mm, less than or equal to 100 µm, less than or equal to 10 µm, less than or equal to 1 µm, less than or equal to 100 nm, less than or equal to 10 nm, less than or equal to 1 nm, etc. Combinations are also possible, e.g., less than or equal to 100 mm and greater than or equal to 10 mm, less than or equal to 10 mm and greater than or equal to 10 µm. Other ranges are possible. For embodiments with 3 or more bioelectrical sensors, each may independently have the same or different spacing from an adjacent bioelectrical sensor.

In addition, two or more bioelectrical sensors may be strategically spaced to optimize the acquisition of data. For instance, in some cases, it may be necessary to simultaneously obtain data at different portions of bodily tissue to generate more accurate and reliable readings. As a hypothetical example, three temperature sensors may be sufficient to measure bodily tissues with consistent temperature (e.g., chest) whereas more than five temperature sensors may be necessary to measure bodily tissues with larger temperature gradients (e.g., arms).

In some embodiments, conductive threads 113 may be incorporated into the textile patches (or into at least a portion of the textile patches). Conductive threads may, in some cases, refer to threads capable of conducting electricity. In some cases, conductive threads may be made of organic materials (e.g., carbon nanotubes) and/or inorganic materials (e.g. silver). In some embodiments, the conductive threads comprise stainless steel. In addition, it should be understood that conductive threads may refer to threads where at least a portion of the material may be electrically conductive. In some cases, the electrically conductive portion may just be the surface of the conductive thread. This may be advantageous for multiple reasons, e.g., to fabricate lighter conductive threads and/or to reduce costs associated with fabricating the conductive threads.

It should also be understood that conductive threads 113 may be present in multiple components of the wearable system and may be configured to perform different functions. In some cases, the conductive threads may be responsible for maintaining certain components (e.g., computing unit and textile patches) in electrical communication with each other. In some cases, the conductive threads may transmit power from the power source into any of the components in the wearable system (e.g., bioelectrical sensors). In some cases, the conductive threads may be associated with one or more components configured to generate and/or read a signal (e.g., an oscillatory signal). For instance, the conductive threads may be able to transmit and/or generate a bioimpedance signal at a portion of the skin tissue and read this signal at another portion of the skin tissue. In addition, in some cases, conductive threads may be part of a thermal sensor, e.g., a thermocouple using graphite-based conductive threads.

In some embodiments, the conductive threads may be a dry electrode 332. Dry electrodes may consist of an electrode in electrical communication with bodily tissue without needing skin abrasions or gels and without causing discomfort to the user. The conductive threads may be strategically placed to maximize skin contact and decrease electrical contact resistance.

In some embodiments, the conductive threads may be used as a conductive pathway to send and/or receive electrical signals from the dry electrodes 332 to the computing unit 111 in the form of electrode lines 336. In other embodiments, the electrode lines may transmit or receive other data and/or electrical signals from bioelectrical sensors (e.g. motion sensors, temperature sensors, etc.) in the form of sensor lines 335.

In some embodiments, at least a portion of the conductive threads 113 may be covered with an insulating material. The insulating material may be an insulating polymer (e.g., polyvinyl chloride, polyethylene, polyvinylidene fluoride, etc.) or an insulating coating (e.g., a dielectric coating). This insulating material may be useful, for example, when conductive threads may cause shorting across the skin and/or when other components may interfere with the function of conductive threads.

The wearable system described herein may comprise a computing unit 111. In some embodiments, the computing unit may comprise a single control module or a plurality of control modules (e.g. microcontrollers), a permanently attached or reversibly attachable memory storage device (e.g. non-voltaile memory), printed circuit boards (PCBs), a power source, a wireless charging interface, and associated hardware to obtain bioelectrical impedance measurements, temperature measurements, heart rate measurements, and/or motion capture data. The associated hardware to obtain bioelectrical impedance measurements may comprise integrated circuits (ICs) designed to produce and measure oscillatory electrical signals applied for skin or body impedance measurements (e.g. Analog Devices AD5940, Analog Devices 1557). Other ICs and/or hardware components may also be used.

The computing unit 111 may further comprise connectors and/or ports (e.g. USB-A, USB-B, USB-C, etc.) to interface with an external device (e.g. cell phone, tablet, computer, etc.) and/or with other bioelectrical sensors via wired communication. The computing unit may comprise a wireless interface for communication (e.g. Bluetooth, Bluetooth Low Energy, Wi-Fi, 4G/5G connectivity, etc.) with external devices and/or other bioelectrical sensors. Using the wired connection and/or the wireless interface, the computing unit may transmit data, physiological metrics, and/or motion tracking information collected from the bioelectrical sensors to one or more external devices.

In some embodiments, the computing unit 111 may comprise a single control module or a plurality of control modules wherein the control module comprises a microcontroller. The microcontroller may interact with the associated hardware to receive and/or process raw data at a particular rate. The microcontroller may send requests to one or many bioelectrical sensors within the wearable system and/or the associated hardware to collect data at a particular point in time. The data may then be sent via conductive threads and/or an electrically conductive pathway (e.g. a metallic wire) in the form of an electrical signal to the microcontroller in a continuous manner or in batches at a particular frequency. The rate at which the microcontroller requests data collection from the bioelectric sensors may be known as a sampling rate. In some embodiments, the sampling rate may be greater than or equal to 1 Hz, greater than or equal to 2 Hz, greater than or equal to 3 Hz, greater than or equal to 4 Hz, greater than or equal to 5 Hz, greater than or equal to 6 Hz, greater than or equal to 8 Hz, greater than or equal to 10 Hz, greater than or equal to 12 Hz, greater than or equal to 14 Hz, greater than or equal to 16 Hz, greater than or equal to 18 Hz, greater than or equal to 20 Hz, or more. In some embodiments, the sampling rate may be less than or equal to 20 Hz, less than or equal to 18 Hz, less than or equal to 16 Hz, less than or equal to 14 Hz, less than or equal to 12 Hz, less than or equal to 10 Hz, less than or equal to 8 Hz, less than or equal to 6 Hz, less than or equal to 5 Hz, less than or equal to 4 Hz, less than or equal to 3 Hz, less than or equal to 2 Hz, less than or equal to 1 Hz, or less. Other ranges are possible.

In some embodiments, the microcontroller may receive data from the bioelectrical sensors and impart various digital signal processing (DSP) methods onto the data to acquire, isolate, analyze, detect, and/or extract waveform characteristics of each bioelectric sensor output. In some embodiments, the microcontroller may correlate variations in the bioelectrical impedance sensor output to body temperature, heart rate, and motion tracking information from each independent axis of bodily motion. The resulting correlation may provide insight into the external reasons of said variations. In some embodiments, the microcontroller may implement pattern recognition algorithms, machine learning algorithms, and/or sensor fusion algorithms onto the correlated dataset to identify physiological trends, reduce uncertainty associated with sensor output, and/or adjust for sensor output variations that may occur when operating on different users. In some embodiments, the user of the wearable system and/or other personnel (e.g., coaches, parents, physicians, clinical staff, etc.) may input data directly into the computing unit or into an external device in communication with the computing unit. The inputted data may then be correlated with acquired data from the bioelectrical sensors. In some embodiments, the inputted data comprises dietary information, physical activity information, sleep information, emotional status information, medication information, and/or other health related information.

In some embodiments, the computing unit 111 may comprise a memory storage device. In some embodiments, the memory storage may be permanently affixed to the computing unit or reversibly detachable from the computing unit. In some embodiments, the memory storage device comprises non-volatile memory. The memory storage device may store raw data from the bioelectrical sensors, data that has been correlated and/or processed by the computing unit as described above, and/or specific characteristics of a waveform that is unique to a particular user. Specific characteristics of the waveform may obtained in a time domain or in a frequency domain via Fourier analysis.

The computing unit 111 may be reversibly detachable from the garment and/or the wearable system. When used in conjunction with the reversibly detachable textile patches as previously described, the associated garment (e.g. a shirt) may be replaced with another garment (e.g. a compressive sleeve) after removal of the computing unit and textile patches. The computing unit and textile patches may then be reattached to render the wearable system functional. The reversibly detachment computing unit may allow for garments to be changed or replaced at the user’s convenience. Advantageously, it also allows the computing unit to be replaced, repaired, or modified without altering or replacing the entire garment or wearable system. In some embodiments, the computing unit comprises of a clip rail that grips onto the fabric of the garment and secures electrical contacts with conductive thread or a conductive pathway (e.g. a metallic wire) that connects to the bioelectrical sensors. The clip rail ensures the computing unit may receive and send electrical signals and/or power to the textile patches and/or bioelectrical sensors. In some embodiments, the computing unit is permanently attached to the garment.

The embodiments described herein may be implemented in any of numerous ways. For example, the embodiments may be implemented by any suitable type of analog and/or digital circuitry. In some embodiments, the embodiments may be implemented using hardware or a combination of hardware and/or software (e.g., a computing unit). When implemented using software, suitable software code may be executed on processing circuitry including any suitable processor (e.g., a microprocessor) or collection of processors, whether provided in a single computer or distributed among multiple computers (or other consumer electronic devices). It should be appreciated that any component or collection of components that perform the functions described above may be generically considered as one or more controllers that control the above-discussed functions. The one or more controllers may be implemented in numerous ways, such as with dedicated hardware or with one or more processors programmed using microcode or software to perform the functions recited above. The one or more embodiments may be implemented in numerous ways, such as with dedicated hardware, or with general purpose hardware (e.g., one or more processors) that is programmed using microcode or software to perform the functions recited above.

In some embodiments, the embodiments described herein comprise wireless capabilities for enabling suitable communication with other devices/systems (e.g., for controlling aspects of the electronic component(s), controlling a source of electromagnetic radiation, controlling a sensor or other component). Wireless devices are generally known in the art and may include, in some cases, LTE, WiFi and/or Bluetooth systems. In some embodiments, the systems and/or devices described herein comprise such a wireless device.

In some embodiments, the embodiments described herein may be configured to adjust various parameters based on external metrics. For example, in some embodiments, the system is configured to adjust the rate, wavelength, pulse, modulation, intensity, etc. of electromagnetic radiation from the source of electromagnetic radiation (e.g., in response to a signal from a sensor and/or consumer electronic device in electrical or wireless communication with and/or associated with the system). In some embodiments, the system adjusts the rate, wavelength, pulse, modulation, intensity, etc. of electromagnetic radiation from the source of electromagnetic radiation in response to an input from the user and/or a signal from the sensor and/or consumer electronic device.

Any electronic component circuitry may be implemented by any suitable type of analog and/or digital circuitry. For example, the electronic component circuitry may be implemented using hardware or a combination of hardware and software. When implemented using software, suitable software code may be executed on any suitable processor (e.g., a microprocessor) or collection of processors. The one or more electronic components may be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, or other tangible, non-transitory computer-readable storage medium) encoded with a computer program (i.e., a plurality of executable instructions) that, when executed on one or more processors, performs the above-discussed functions of one or more embodiments. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that may be employed to program one or more processors to implement aspects of the techniques discussed herein.

Optimization may involve, in some embodiments, creating a set of parameters that maximizes the signal and/or minimizes (suppresses) the background (e.g., including stray light) signals. These parameters generally depend on the particular assay and conditions under which the reading is conducted.

In some embodiments, the wearable system comprises a single power source or a plurality of power sources. In some embodiments, a computing unit comprises the power source or a plurality of power sources. In some cases, the power source comprises a portable electrochemical battery (e.g. a lithium-ion battery). In some embodiments, the power source comprises an electrochemical battery that may be recharged when depleted of charge. The power source may be recharged via a wired recharging connector or recharged wireless through a wireless charging interface via local electromagnetic coupling. In some embodiments, the computing unit comprises a wireless charging interface wherein the wireless charging interface is an inductive wireless charging coil. In some embodiments, the power source is in electrical communication with the microcontroller and/or a plurality of bioelectrical sensors. The power source may supply power to the bioelectrical sensors and to the microcontroller so that they may function as designed. In some embodiments, a wearable system comprises a photovoltaic cell or a plurality of photovoltaic cells to supply power, generated from ambient light or sun light to the microcontroller and bioelectrical sensors.

The power source may include any appropriate material(s), such as one or more batteries, photovoltaic cells, etc. Non-limiting examples of suitable batteries include Li-polymer (e.g., with between 100 and 1000 mAh of battery life), Li-ion, nickel cadmium, nickel metal hydride, silver oxide, or the like. In some cases, the battery may apply a voltage in response to a physiological and/or external metric and/or signal (e.g., by a user). For example, the voltage may be used to trigger the exit of the resident structure by e.g., applying a voltage to thermally sensitive degradable component as described herein. For example, the average magnitude of the voltage applied may be between 0.001 to 0.01 V, between 0.01 to 0.1 V, between 0.1 V and 10.0 V, between 1.0 V and 8.0 V, between 2.0 V and 5.0 V, between 0.1 V and 5.0 V, between 0.1 V and 1.5 V, between 0.1 V and 1.0 V, between 1.0 V and 3.0 V, between 3.0 V and 8.0 V, or any other appropriate range.

The wearable system described herein comprises textile patches, bioelectrical sensors, conductive thread, a computing unit, and/or a power source and is configured to be worn by a user. In some embodiments, the wearable system may be integrated into a garment to be worn by a user. The garment may be a shirt, pants, compression shorts, back braces, girdles, compression sleeves, shoes, socks, jackets, masks, headbands, sweatbands, wristbands, and/or hats. Other garments are also possible. Multiple garments comprising various embodiments of the wearable system may be worn simultaneously. For example, a shirt comprising a wearable system may be worn in conjunction with a sweatband comprising a wearable system. The wearable system may be portable and comfortably worn and/or transported by the user due to the use of soft, flexible, and elastic materials.

The wearable system may be configured to collect a plurality of determinable signals from the plurality of bioelectrical sensors. As previously described, there may be multiple bioelectrical sensors per garments and multiple garments may be worn by the user at a given time. The wearable system may collect a plurality of determinable signals from the sensor output of a single bioelectrical sensor or multiple bioelectrical sensors per instructions implemented by the computing unit. The plurality of determinable signals may be correlated with a physiological health metric of the user using DSP, machine learning methodologies, pattern recognition methodologies, and/or sensor fusion methodologies, as previously described. Other methodologies, algorithms, and strategies for generating general physical health metrics of the user from the plurality of determinable signals are also possible. General physical health metrics comprise hydration, nutrition, body fat percentage, water mass percentage, skeletal mass percentage, muscular activity, muscular fatigue, sleep quality, and/or injury prevention. Other general health metrics are also possible.

In some embodiments, the user may comprise an animal or human e.g., capable of carrying out physical or physiological activity. In some embodiments, physiological metrics or general health metrics may be cross correlated with each other or with data the user inputs into the wearable system to produce new metrics. For example, if the user inputs information about recently ingested items, the wearable system may correlate the inputted data with the hydration data collected from the bioelectrical sensors to produce a general nutrition metric encompassing nutrient intake from solid and liquid nutrient sources.

In some embodiments, the wearable system may power down into a standby state or turn off upon removal from the user to conserve energy of the power source. The wearable system may adjust the power consumption of the microcontroller and bioelectrical sensor to the activity of the user. For example, when the user is highly active, the computing unit may increase the sampling rate and data correlating speeds, but when the user is in a rest state, the computing unit may decrease the sampling rate and data correlating speeds in an effort to conserve energy of the power source.

To reduce the cost of the wearable system, as previously disclosed, the textile patches and computing unit may detach from the garment. The resulting garment comprising conductive thread and attachment rails for the computing unit may reduce the total cost of the garment. The computing unit and textile patches may be reused and placed on different garments (e.g. shirts, pants, socks, etc.) thus limiting the number of textile patch and computing unit purchases for the purchaser. Compatible garments may therefore also produced at a reduce cost as compatible garments may not require, in some embodiments, a dedicated computing unit or dedicated textile patches. Since the textile patches, the computing unit, and/or the power source may detach from the garment, the garment may be machine washed without loss of functionality of the wearable system once dry. In some embodiments, the wearable system comprises encapsulants that render electronic components (ICs, batteries, circuit elements, etc.) water-resistant and/or capable of being machine-washed without damage being incurred to said electronic components .

The wearable system described herein comprises additional stitching, fasteners (e.g., buttons, buckles, clasps, ties, grommets, zippers, snaps, magnets, pins, hook-and-loop), adhesives, and/or other clips to reversibly attach the computing unit, the textile patches, and/or the power source to the garment. Other attachment mechanisms may also be used.

In some embodiments, the wearable system may be used to determine muscle fatigue through various methods. In some cases, an oscillatory electric signal (e.g., a bioimpedance signal) may be applied through the skin and across one or more muscles. The physiological state (e.g., muscle fatigue) of one or more muscles may be determined based on the response of the bioimpedance signal.

The wearable system described herein may be used in several applications. In some embodiments, the wearable system may detect health abnormalities of the user (e.g. hyperextension of joints, muscle/joint overuse risks, heart rhythm abnormalities, dehydration, etc.). After detection, the wearable system may alert or send a warning to the user or other associated personnel of the health abnormalities directly or via the external device. The alerts or warnings may assist the user in preventing future injuries. For example, the wearable system may alert the user if a particular muscle or muscle group is experiencing fatigue after a particular activity, increasing its risk of failure or severe injury. In some embodiments, the wearable system may alert or send a warning to the user of existing or new injuries or health emergencies (e.g. seizures, heart attacks, hypoglycemic episodes, hyperglycemic episodes, ligament tears, tendon tears, muscle strains, joint dislocations, etc.). The user, now equipped with this alert, may reduce the intensity of the activity, or cease performing the activity altogether thereby limiting the risk of injury. Other associated personnel may comprise physicians, clinical staff, coaches, parents, and/or other parties with an interest in the user’s health and physical status. In some embodiments, the wearable system may inform the user of their muscular response to a particular activity relative to the user’s total muscle capacity and/or their response speed of a particular muscle group. In some embodiments, the wearable system may acquire a tissue composition analysis from the user. The tissue composition analysis may provide the user with information regard their body fat percentage, muscle mass percentage, and water mass percentage.

The wearable system described herein may provide the user with a hydration metric indicating the state of hydration of the user. The hydration metric may provide recommended actions for the user to take. For example, the wearable system may recommend to the user to increase their water intake, increase their electrolyte intake, or seek medical attention. Other recommendations may be possible. In some embodiments, the wearable system may incorporate user inputted data regarding the user’s diet. For example, the user may input details regarding recently ingested food, such content and quality, via a connected external device. The wearable system may correlate this information with the hydration metric to provide the user an overall nutrition metric. The nutrition metric may include certain quantitative sub-metrics including but not limited to total calories, net calories, mass of water intake, mass of ingested protein, mass of ingested fat, mass of ingested carbohydrate, mass of ingested fiber, mass of ingested vitamins, and mass of ingested minerals. Other sub-metrics may be possible. In some embodiments, the wearable system may provide a sleep quality assessment to the user. When asleep, the wearable system may track and monitor the quality and stage of sleep (e.g. wake, N1, N2, N3, Restless Eye Movement (REM)). In some embodiments, the wearable system may alert the user via an external device when changes in sleep quality, abnormal sleep quality, or sleep deprivation is detected. In some embodiments, the wearable system may alert the user via a haptic or vibrational tactile device or an external device or directly to wake the user during a lighter sleep phase.

The wearable system may be used as a tool for motion control of robotics, prostheses, and/or motion tracking for virtual reality and/or augmented reality applications. For example, the user may wear the wearable system and move in a physical manner, and the wearable system may capture or storage the physical movements. The movements then may be partially replicated, fully replicated, or mimicked on a robotics platform, a prostheses platform, and/or in a virtual reality or augmented reality platform. Other platforms are also possible. In some embodiments, the wearable system may replay or reenact the physical movements on an external device via a 3D model. The user may use this information to alter, change, or improve the future physical movements. For example, a user may wear the wearable system during a sporting event. After the event, the user may view a three-dimensional rendering of their movement throughout the event. After viewing, the wearable system make recommend adjustments to the user’s movement strategies to improve their performance in future sporting events.

In some embodiments, muscle activity may be monitored or otherwise determined for short, repetitive actions, such as running or weightlifting. However, in some embodiments, muscle activity may be monitored over longer time periods, such as over a culmination of a series of exercises, or trends over days, weeks, months, or longer.

In some embodiments, a wearable health sensing system is described comprising a detachable computing unit or integrated computed unit containing the majority of electrical components such as battery, charging circuitry, microcontroller, and bioelectrical sensors, a rechargeable battery energy source, a wireless interface for local electromagnetic recharging of battery systems, a memory storage device including non-volatile memory that may be attached to and removed from the computing unit, a wireless or wireline interface for communication of data to a remote computing resource or to obtain configuration data or program executable data from a remote computing resource, a plurality of textile patches configured to act as dry electrodes 332 that may be applied separately from garments, a plurality of conductive patches that may be integrated with garments, a plurality of acceleration and gyroscope motion and temperature sensors 334, a plurality of conductive thread or filament capable of powering and/or communicating data and/or electrical signals between various textile patches, sensors and the computing unit whereby from the use of the technology integrated into some form of wearable clothing the user may (a) measure or detect physiological health metrics such as fatigue, hydration, nutrition, sleep deprivation, muscular response, injuries, and provide health warnings. The same device may also (b) be used to provide in-depth analysis of muscular response and movement in an attempt to track the user’s precise bodily motions to control some form of computer program or physical robotics application such as a prosthetic.

In some embodiments, the wearable system also includes a scalable dry electrode compromising conductive fabric, conductive filaments, whereby the fabric is integrated into wearable garments to provide a dry electrical connection to the user’s body. This integration is targeted to certain regions of a garment that experience a tighter fit or are likely to experience skin contact given normal use. While insulated, the filament acts to carry electrical signals across a garment with no shorting across the skin. With the insulation removed, the conductive filament is sewn into the conductive fabric patch to provide an electrical connection to the large conductive area with minimal resistance. Insulated conductive filament connects the electrodes to the computing unit which contains pertinent sensing hardware.

In some embodiments, the wearable system includes a modular sensor circuit compromising an accelerometer / gyro integrated circuit, thermometer integrated circuit, conductive filament, whereby the sensing hardware is integrated into key locations of a garment to actively track bodily movement and temperature. The individual integrated circuits are attached between layers of fabric and held in place via stitching and other means. Insulated conductive filament connects the integrated circuits to the computing unit.

In some embodiments, the wearable system is a garment-integrated device that is machine-washable. Depending on the configuration the computing unit may be detachable for charging and washing. The rest of the device is made using machine-washable components. The conductive fabric and filaments are rated for machine washing. The integrated motion and temperature sensors are waterproofed, layered in a fabric and are able to be machine-washed.

In some embodiments, an algorithm compromising the following components is described: data sampling from bioelectrical, motion, temperature, and other sensors, digital signal processing (DSP), pattern recognition, machine learning, sensor fusion whereby the bioelectrical signals undergo DSP to provide waveform characteristics. The motion data gathered from each independent axis of bodily motion is processed and correlated to variations in bioelectrical activity. This correlation aids in the processing of the bioelectrical signals as it provides external reasoning for variations in these bioelectrical signals. After this initial processing the motion and bioelectrical data is integrated with body temperature data. This dataset is then analyzed via pattern recognition algorithms. To adjust for differences in users, there is a machine learning component that tunes the device to provide optimal classifications and metrics.

In some embodiments, the algorithm includes a process of inferring: nutrition characteristics, sleep quality characteristics, whereby the processed data is analyzed for long term trends which are cross correlated with direct user feedback.

In some embodiments, the algorithm includes a process of adapting performance of pattern recognition, machine learning, and sensor fusion methods based on training of algorithms on observation of sensor data, user feedback, and assessment of user state by observers.

In some embodiments, the algorithm includes a process of controlling or manipulating prosthetics, robotics, augmented / virtual reality, whereby the data is processed to track fine bodily movements.

In some embodiments, the algorithm of includes a process using muscular electrical activity and impedance changes whereby muscular fatigue characteristics and muscular injury characteristics may be detected and measured.

In some embodiments, the algorithm includes a method of determining hydration characteristics whereby the metric is directly determined from the processed bioelectrical data in the form of tissue composition analysis.

In some embodiments, the algorithm includes a method of interpreting the acceleration and gyroscope motion data to track the precise movements of the user’s body for use in motion capture applications such as virtual / augmented reality scenes. For example, This could also be used to replay specific movements precisely during or after a sporting practice or event.

### Prophetic Example

Wearable health sensing devices represent a growing portion of consumer electronic devices. These devices generally incorporate motion analysis and/or heart rate monitoring to provide feedback on the general health of the user and provide metrics on the daily activities of the user. What is described herein is an exemplary embodiment of an apparatus capable of finer physiological and health measurements that may be integrated into everyday textiles. This apparatus relies partly on the measurement of bioelectrical characteristics through the use of textile-based sensors. Two examples of well-studied bioelectrical sensor technologies are electromyography and bioelectrical impedance analysis. Electromyography (EMG) is the study of the bioelectrical activity produced by the body’s muscle activations.

Bioelectrical impedance analysis (BIA) uses, in some embodiments, a small oscillating current to be passed through the body upon which unique characteristics of the crossed tissues may be measured by how the signal is impeded. Without wishing to be bound by theory, the combination of bioelectrical characteristics, external temperature and motion data may provide, in some embodiments, a more detailed insight into a user’s physiological state, muscular activity and general physical health than any current wearable device.

The inventive apparatus provides a portable and wearable means of measuring bioelectrical characteristics of body tissue, multi-axis motion and external body temperature to provide detailed and useful metrics. Such metrics may include one or more of health warnings, performance advice, and injury prevention. The apparatus in some embodiments is composed of a circuit board, a plurality of modular sensor packages and configurable textile electrodes that are capable of being embedded into textiles. The combination of such sensing technologies generally allows for in-depth physiological and health sensing. The target metrics include but are not limited to: fatigue, hydration, nutrition, sleep deprivation, muscular response, injury detection, and motion tracking. Target applications may be categorized into the following categories:
Health Monitoring
   Hydration (or Dehydration) Metric
   Muscular Fatigue Monitoring
   Muscular Injury Monitoring
   Nutrition
   Sleep Deprivation
Virtual Tracking
   Motion Capture (for use in VR/AR or other media)
   Prostheses Control
   Robotics Control

These metrics may be tracked internally and communicated to an external device such as the user’s phone or PC via Bluetooth Low Energy. The device may be powered by an internal battery and may be charged via wireless inductive charging. The end device may incorporate, in some embodiments, a singular compute unit along with plurality of conductive textile patches and corresponding temperature and motion sensors. The density of temperature and motion sensors on the body may provide, in some embodiments, a detailed map of movement and body temperature that may be cross-correlated with the bioelectrical measurements taken using the conductive fabric as dry electrodes. These measurements may be fed into proprietary algorithms that make advanced predictions and/or direct assessment of physical health and body movement. All the textile patches may be integrated permanently into the article of clothing. The compute unit may be detachable and replaceable.

An exemplary shirt-based configuration of the apparatus, shown in FIG. 2, is composed of two distinct hardware units using a plurality of electronic components, printed circuit boards (PCBs) and conductive textiles. The first unit may be referred to as the computing unit (111) and is shown in an exemplary configuration in FIG. 3. The computing unit contains, in some cases, the microcontroller and hardware for bioelectrical analysis. This unit may be integrated into the textile or made attachable through the use of a removable and reusable attachment mechanism. The computing unit contains the battery and wireless charging circuitry necessary to operate the device as a whole. Whether the computing unit is attachable or permanently integrated, it powers the entire completed textile when configured properly. The second unit (112) exists in multiple forms but may be generalized as a textile patch that has either conductive thread or conductive fabric integrated into it. Patches of this conductive thread or fabric may be woven to make dry electrodes capable of performing bioelectrical measurements. These electrode pads may be strategically placed to maximize skin contact based on a given article of clothing (e.g. shirt, pants, compression sleeve). The textile patches may also be configured to have small and inexpensive electronic components such as accelerometers, gyros and thermometers integrated directly into them. This exemplary configuration is shown in FIG. 4. By delegating more expensive components involved in processing and circuitry inside the computing unit, the total cost per textile unit is reduced to enable clothing articles that may be manufactured cheaply with the computing device being a one-off per user. An example of a complete device is shown in FIG. 2 where the central device is clipped in near the neck of a shirt. Multiple different configurations of textile patches may be used based on the desired application. FIG. 5 and FIG. 6 show different configurations where the central device and textile patches are integrated into compression shorts and into a compression sleeve respectively.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A wearable system configured to be worn by a user, the system comprising:
a plurality of reversibly detachable textile patches;
a plurality of bioelectrical sensors integrated into each reversible detachable textile patch;
a reversibly detachable computing unit;
a power source in electrical communication with the plurality of bioelectrical sensors; and
a plurality of conductive threads in electrical communication with the reversibly detachable computing unit and the plurality of bioelectrical sensors,
wherein a first bioelectrical sensor of the plurality of bioelectrical sensors is a bioelectrical impedance analysis sensor, and
wherein the wearable system is configured to collect a plurality of determinable signals from the plurality of bioelectrical sensors, wherein the plurality of determinable signals are correlated with a physiological health metric of the user.

2. The system of claim 1, wherein the plurality of bioelectrical signals further comprises a second bioelectrical sensor comprising a temperature sensor and/or a motion sensor.

3. A wearable system for sensing muscle fatigue, the system comprising:
a plurality of textile patches;
a plurality of bioelectrical sensors integrated into the plurality of textile patches, wherein the textile patches are configured to be integrated into a garment;
a computing unit; and
a plurality of conductive threads electrically connecting the computing unit and the plurality of textile patches.

4. The system according to claim 1, wherein the plurality of textile patches are configured to detach from the garment.

5. The system according claim 1, wherein the bioelectrical sensors are permanently integrated in the garment.

6. The system according to claim 1, wherein the system further comprises a portable energy source.

7. The system according to claim 1, wherein the system comprises a portable energy source configured to be recharged wirelessly.

8. The system according to claim 1, wherein the conductive threads are at least partially covered by an electrically insulating material.

9. The system according to claim 1, wherein the computing unit comprises a control module.

10. The system according to claim 1, wherein the system further comprises a storage device that is detachable from the control module.

11. The system according to claim 1, wherein the system has a wireless interface or a wired port to communicate with an external device.

12-13. (canceled)

14. A method for determining muscle fatigue, the method comprising:
providing a plurality of bioelectrical sensors integrated into the plurality of textile patches to a surface of skin;
applying an oscillatory electric signal through the skin and across one or more muscles;
determining a bioimpedance of the oscillatory electric signal;
determining a state of the one or more muscles based, at least in part, on the determining of the bioimpedance.

15. The method of claim 14, wherein a period of oscillation of the oscillatory signal is greater than or equal to 10 kHz and less than or equal to 200 kHz.

16. The method of claim 14, wherein a max/min amplitude of the AC current is greater than or equal to 0.1 µA or less than or equal to 1500 µA.
